(19) United States
(12) Patent Application Publication
(10) Pub. No.: US 2015/0044745 A1
(43) Pub. Date: Feb. 12, 2015

(54) RECOMBINANT CORYNEBACTERIUM AND A METHOD OF PRODUCING C4 DICARBOXYLIC ACID USING THE SAME

(71) Applicant: Samsung Electronics Co., Ltd., Suwon-si (KR)

(72) Inventors: Soonchun CHUNG, Seoul (KR); Joonsong Park, Seoul (KR); Jinhwan Park, Suwon-si (KR); Jiae Yun, Hwaseong-si (KR); Jaechan Park, Yongin-si (KR); Kwangmyung Cho, Seongnam-si (KR)

(21) Appl. No.: 14/456,754

(22) Filed: Aug. 11, 2014

(30) Foreign Application Priority Data
Aug. 9, 2013 (KR) 10-2013-0094885

(51) Int. Cl. C12P 7/46; C12N 9/88

(52) U.S. Cl. CPC C12P 7/46; C12N 9/88; C12Y 402/01002
USPC 435/145; 435/252.32; 435/471

(57) ABSTRACT

A recombinant Corynebacterium genus microorganism, and a method of producing C4 dicarboxylic acid under anaerobic conditions using the Corynebacterium genus microorganism.

### CROSS-REFERENCE TO RELATED APPLICATIONS

This application claims the benefit of Korean Patent Application No. 10-2013-0094885, filed on Aug. 9, 2013, in the Korean Intellectual Property Office, the entire disclosure of which is hereby incorporated by reference.

### INCORPORATION BY REFERENCE OF ELECTRONICALLY SUBMITTED MATERIALS

Incorporated by reference in its entirety herein is a computer-readable nucleotide/amino acid sequence listing submitted herewith and identified as follows: 35,501 bytes ASCII (Text) file named “718134_ST25.TXT,” created Aug. 7, 2014.

### BACKGROUND

1. Field

The present disclosure relates to a recombinant Corynebacterium and a method of producing C4 dicarboxylic acid using the same.

2. Description of the Related Art

Microorganisms of Corynebacterium genus are Gram-positive strains and used for producing amino acids such as glutamate, lysine, and threonine. Corynebacterium glutamicum has advantages as a strain for industrial use since the growth conditions thereof are simple, the genome structure is stable, and the strain is harmless to environment.

Corynebacterium glutamicum is an aerobic bacterium. Under anaerobic conditions wherein oxygen is absent or insufficient to sustain the microorganism, the metabolic processes of Corynebacterium glutamicum are stopped except for metabolic processes involved in producing the minimum energy for survival, as Corynebacterium glutamicum produces and secretes lactic acid, acetic acid, and succinic acid for energy production. If a reductive TCA (tricarboxylic acid cycle) cycle is used under an anaerobic condition, succinic acid is produced from oxalacetic acid by malate dehydrogenase (mdh), fumarase, and succinate dehydrogenase complex (sdhCAB).

Fumarase is an enzyme that catalyzes the conversion of a substrate such as malate or fumarate to fumarate or malate, respectively. While Escherichia coli has three types of fumarase, that is, fumarase A, fumarase B, and fumarase C, Corynebacterium glutamicum is known to have only one type of fumarase.

A TCA cycle is a metabolic process occurring in all biological species, wherein energy and metabolic intermediates are produced. The metabolic intermediates of a TCA cycle, such as succinic acid, fumarate, and malate are biosynthesized into useful chemicals through various metabolic processes. A method is needed in which the production of such TCA cycle metabolic intermediates using Corynebacterium glutamicum under anaerobic conditions is increased.

### SUMMARY

Provided is a Corynebacterium genus microorganism including a gene encoding a fumarase polypeptide having a higher substrate affinity to malate than to fumarate. Also provided is a method of producing C4 dicarboxylic acid using the microorganism.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic diagram representing substitution of a fumC gene with a fumB gene.

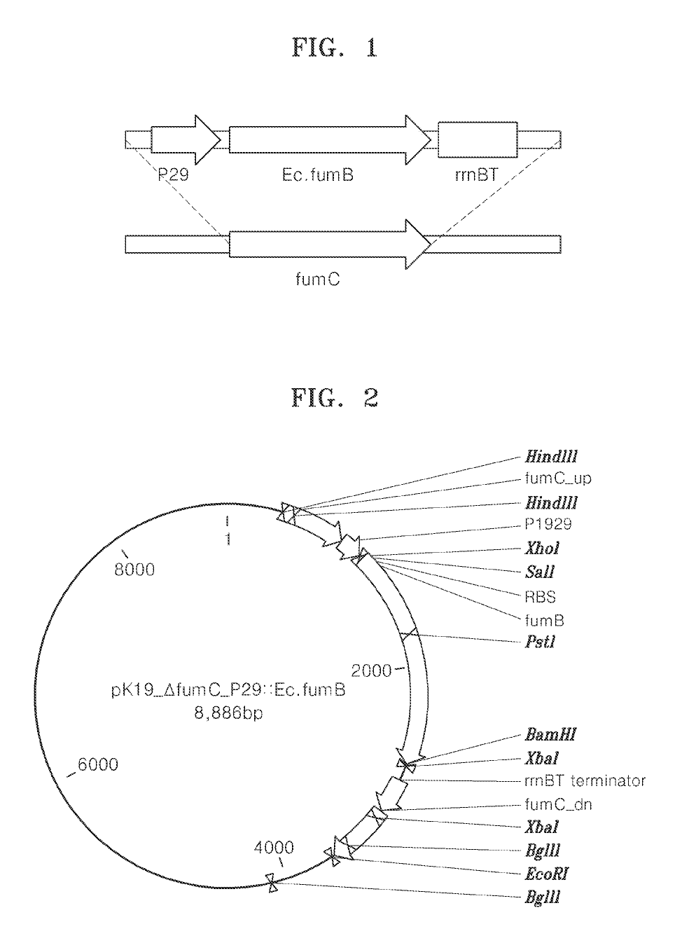

FIG. 2 is a restriction map of a pK19_ΔfumC_P29::Ec.fumB recombinant vector.

FIGS. 3A, 3B, and 3C are bar graphs showing the culture results of a Corynebacterium microorganism in which the endogenous fumC gene was substituted with a fumB gene.

### DETAILED DESCRIPTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein.

Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

An aspect of the present disclosure provides a Corynebacterium genus microorganism including a gene encoding a fumarase polypeptide having a higher substrate affinity to malate than to fumarate.

The term “fumarase polypeptide” refers to an enzyme that serves as a catalyst for converting malate to fumarate. The fumarase polypeptide may be an enzyme classified as EC 4.2.1.2. Substrate affinity may be represented by a Michaelis-Menten constant (Kₘ). The Kₘ value of the fumarase polypeptide to malate may be smaller than the Kₘ value of the fumarase polypeptide to fumarate. The fumarase polypeptide may be fumarase B. The fumarase polypeptide may be another fumarase polypeptide having a higher substrate affinity to malate than to fumarate. The fumarase B may include an amino acid sequence having a sequence identity of 70% or higher with the amino acid sequence of a fumarase derived from Escherichia coli, Shigella dysenteriae, S. flexneri or S. boydii. The fumarase B may include an amino acid sequence having a sequence identity of 70% or higher with SEQ ID NO.1, for example, 75% or higher, 80% or higher, 90% or higher or 95% or higher. The fumarase B may include an amino acid sequence of SEQ ID NO.1. The gene encoding the fumarase polypeptide having a higher substrate affinity to malate than to fumarate may include a nucleotide sequence of SEQ ID NO.2.

The term “sequence identity” of a nucleic acid or a polypeptide herein means the degree of identity between bases or amino acid residues as two sequences therein are aligned in a specific comparison region in a way that the sequences may be matched up with each other at as many bases or amino acid residues as possible. Sequence identity is measured by optimally aligning two sequences in a specific comparison region and comparing the identity between the two sequences. A part of one sequence in a specific comparison region may be added or deleted in comparison to a reference sequence. Percent sequence identity may be calculated in a step of yielding percentage of sequence identity by determining the number of positions in which nucleic acid bases or amino acid residues are identical in a comparison region of two sequences, dividing the number of the identical positions by the total number of positions, and multiplying the result by 100. The percent sequence identity may be determined by using known sequence-comparing software programs such as BLASTn (NCBI) and MegAlign™ (DNASTAR Inc). Various levels of sequence identity may be used to identify many polypeptides or genes having an identical or similar function or activity. For example, a percent sequence identity of 50%, 55%, 60%, 65%, 70%, 75%, 80%, 85%, 90% or 95% may be used.

The genes may or may not be inserted in a chromosome. The genes may be introduced into the chromosome by a vehicle such as a vector. The vector may include a regulatory sequence and/or a homologous region operably linked with the genes. The term “operably linked” herein mean a functional bond between a regulatory sequence of a nucleic acid expression and another nucleotide sequence. Therefore, the regulatory sequence regulates transcription and/or translation of the genes. The regulatory sequence may include a promoter, a terminator, a ribosome biding site, an enhancer or a combination thereof. The promoter may be, for example, a NCgl1929 promoter, a tuf promoter, or a tac promoter. The terminator may be, for example, an rrnB terminator. A homologous region is a region recognized by a recombinase, and cross-linked with a chromosome. A homologous region may be positioned or inserted upstream and/or downstream from the polynucleotide to be inserted. Insertion of a gene into a chromosome may be performed by homologous recombination. For example, a gene may be inserted into a chromosome by inducing an insertion of the gene into the chromosome through homologous recombination by preparing a recombinant vector by inserting the gene into a vector and then introducing the recombinant vector to a microorganism.

In a Corynebacterium genus microorganism having a gene encoding a fumarase polypeptide having a higher substrate affinity to malate than to fumarate, endogenous fumarase activity may be eliminated or decreased. Endogenous fumarase activity refers to the activity of fumarase proteins that are native to the microorganism. The term “decreased” may represent relative activity of the genetically engineered microorganism in comparison to activity of a microorganism of the same type which is not genetically engineered. The endogenous fumarase may have a higher substrate affinity to fumarate than to malate. The Kₘ value of the endogenous fumarase to fumarate may be smaller than the Kₘ value of the endogenous fumarase to malate. The endogenous fumarase may be fumarase C. The fumarase C may have an amino acid sequence of SEQ ID NO: 3.

In a Corynebacterium genus microorganism having a gene encoding a fumarase polypeptide having a higher substrate affinity to malate than to fumarate, an endogenous fumarase gene may be inactivated or attenuated. The term “inactivation” may mean that a gene is not expressed or a gene is expressed but a product of the expressed gene is not active. The term “attenuation” may mean that the expression of a gene is decreased to a level lower than an expression level of wild type strain, a strain which is not genetically engineered, or a parent strain (e.g., the strain from which the recombinant Corynebacterium having a gene encoding a fumarase polypeptide having a higher substrate affinity to malate than to fumarate is produced). Alternatively, “attenuation” may mean that a product of the expression of the gene has a decreased activity. The inactivation or attenuation may be performed, for example, by homologous recombination. The inactivation or attenuation may be performed, for example, by transforming a vector including a part of the sequence of the genes into a cell, culturing the cell so that homologous recombination of the sequence may occur with a homologous gene of the cell, and then using a selection marker to select a cell in which homologous recombination has occurred. The endogenous fumarase gene may have a nucleotide sequence of SEQ ID NO: 4.

In a Corynebacterium genus microorganism having a gene encoding a fumarase polypeptide having a higher substrate affinity to malate than to fumarate, a pathway in which lactate is synthesized from pyruvate may be inhibited or blocked. In the microorganism, activity of L-lactate dehydrogenase (LDH) may be eliminated or decreased. In the microorganism, a gene encoding LDH may be inactivated or attenuated. The LDH may be an enzyme classified as EC.1.1.1.27. The LDH may have an amino acid sequence of SEQ ID NO: 5 or that having a sequence identity of 70% or higher with SEQ ID NO: 5.

In addition, in a Corynebacterium genus microorganism having a gene encoding a fumarase polypeptide having a higher substrate affinity to malate than to fumarate, a pathway in which acetate is synthesized from pyruvate may be inhibited or blocked. In the microorganism, activity of at least one protein selected from the group consisting of puruvate oxidase (PoxB), phosphotransacetylase (PTA), acetate kinase (AckA), and acetate coenzyme A transferase (ActA) may be eliminated or decreased. The PoxB may be an enzyme classified as EC.1.2.5.1. The PoxB may have, for example, an amino acid sequence of SEQ ID NO: 6 or that having a sequence identity of 70% or higher with SEQ ID NO: 6. The PTA may be an enzyme classified as EC.2.3.1.8. The PTA may have, for example, an amino acid sequence of SEQ ID NO: 7 or that having a sequence identity of 70% or higher with SEQ ID NO: 7. The AckA may be an enzyme classified as EC.2.7.2.1. The AckA may have, for example, an amino acid sequence of SEQ ID NO: 8 or that having a sequence identity of 70% or higher with SEQ ID NO: 8. The ActA may be an enzyme classified as EC.2.8.3.8. The ActA may have, for example, an amino acid sequence of SEQ ID NO: 9 or that having a sequence identity of 70% or higher with SEQ ID NO: 9. In the microorganism, at least one gene selected from the group consisting of a gene encoding PoxB, a gene encoding PTA, a gene encoding AckA, and a gene encoding ActA may be inactivated or attenuated. The term “gene” herein may include a region encoding a protein or the combination of a region encoding a protein and a region regulating expression of the protein.

The microorganism may be a microorganism selected from the group consisting of Corynebacterium glutamicum, Corynebacterium thermoaminogenes, Brevibacterium flavum, and Brevibacterium lactofermentum.

Another aspect of the present disclosure provides a method of preparing C4 dicarboxylic acid including a step of culturing a recombinant Corynebacterium genus microorganism having a gene encoding a fumarase polypeptide having a higher substrate affinity to malate than to fumarate; and a step of recovering C4 dicarboxylic acid from the culture.

The recombinant Corynebacterium genus microorganism is described in this disclosure.

The culturing of the microorganism may be performed in an appropriate culture medium and according to conditions appropriate for culturing as known in the concerned industry. The culturing procedure may be adjusted according to the selected microorganism. The culturing method may include batch culturing, continuous culturing, fed-batch culturing or a combination thereof.

The culture medium may include various carbon sources, nitrogen sources, and trace elements.

The carbon source may include a carbohydrate such as glucose, sucrose, lactose, fructose, maltose, starch, and cellulose; a lipid such as soybean oil, sunflower oil, castor oil, and coconut oil; a fatty acid such as palmitic acid, stearic acid, and linoleic acid; an organic acid such as acetic acid; or a combination thereof. The culturing may be performed by using glucose as a carbon source. The nitrogen source may include an organic nitrogen source such as peptone, yeast extract, meat extract, malt extract, corn steep liquid, and soybean; an inorganic nitrogen source such as urea, ammonium sulfate, ammonium chloride, ammonium phosphate, ammonium carbonate, and ammonium nitrate; or a combination thereof. The culture medium may include as a phosphorous source, for example, potassium dihydrogen phosphate, dipotassium phosphate, a sodium-containing salt corresponding to potassium dihydrogen phosphate, and dipotassium phosphate, and a metal salt such as magnesium sulfate and iron sulfate. The culture medium or an individual component may be added to the culture in a batch mode or a continuous mode. The preferred culture medium may brain heart infusion-supplemented (BHIS) (e.g., having the composition as described in Example 1), CGXII medium (e.g., including 20 g/L (NH₄)₂SO₄, 5 g/L urea, 1 g/L KH₂PO₄, 1 g/L K₂HPO₄, 0.25 g/L MgSO₄.7H₂O, 10 mg/L CaCl₂, 10 mg/L FeSO₄.7H₂O, 0.1 mg/L MnSO₄.H₂O, 1 mg/L ZnSO₄.7H₂O, 0.2 mg/L CuSO₄.5H₂O, 20 mg/L NiCl₂.6H₂O, 0.2 mg/L biotin, 42 g/L 3-(N-morpholino)propanesulfonic acid (MOPS), and 4% (w/v) glucose), 51 medium (e.g., having the composition as described in Example 3), SF1 medium (e.g., having the composition as described in Example 3) or a combination thereof.

In addition, pH of the culture may be adjusted during the culturing by adding a compound such as ammonium hydroxide, potassium hydroxide, ammonia, phosphoric acid or sulfuric acid to the culture in an appropriate mode. In addition, bubble formation may be repressed by using a defoaming agent such as fatty acid polyglycol ester.

The microorganism may be cultured under anaerobic conditions. An anaerobic condition may be formed, for example, by supplying carbon dioxide or nitrogen at a flow rate range from about 0.1 vvm (aeration volume/medium volume/minute) to about 0.4 vvm, from about 0.2 vvm to about 0.3 vvm, or at a flow rate of about 0.25 vvm. The culturing temperature may be in the range from about 20° C. to about 45° C. or from about 25° C. to about 40° C. The culture duration may be extended until a desired amount of target C4 dicarboxylic acid is acquired.

The C4 dicarboxylic acid may be an acid having a carbon number of 4 and two carboxylic groups, or a salt of the acid. For example, the C4 dicarboxylic acid may be malate, fumarate, or succinic acid.

The recovery of the C4 dicarboxylic acid may be performed by using a known separation and purification method. The recovery may be performed by centrifugation, ion exchange chromatography, filtration, precipitation, or a combination thereof.

Hereinafter, embodiments of the present disclosure are described in detail with reference to Examples, but embodiments of the present invention are not limited thereto.

### EXAMPLE 1

### Preparation of a Strain in Which the Lactate and Acetate Synthesis Pathways are Eliminated and Comparison of Intracellular Malate and Fumarate Quantity

(1) Preparation of Replacement Vector

L-lactate dehydrogenase (ldh), pyruvate oxidase (poxB), phosphotransacetylase (pta), acetate kinase (ackA), and acetate CoA transferase (actA) genes of Corynebacterium glutamicum (C. glutamicum, CGL) ATCC 13032 were inactivated by homologous recombination. To inactivate the genes, pK19 mobsacB (ATCC 87098) vector was used as follows.

Two homologous regions for the elimination of the ldh gene were located upstream and downstream from the gene and obtained by PCR amplification using a primer set including IdhA_{—}5′_HindIII (SEQ ID NO: 10) and IdhA_up_{—}3′_XhoI (SEQ ID NO: 11) and a primer set including IdhA_dn_{—}5′_XhoI (SEQ ID NO: 12) and IdhA_{—}3′_EcoRI (SEQ ID NO: 13). The PCR amplification was performed by repeating, 30 times, a cycle including a denaturation step at 95° C. for 30 seconds, an annealing step at 55° C. for 30 seconds, and an extension step at 72° C. for 30 seconds. All the PCR amplifications hereinafter were performed under the same conditions. A pK19_Δldh vector was prepared by cloning the obtained amplification product to the HindIII and EcoRI restriction enzyme positions of pK19 mobsacB vector.

Two homologous regions for the elimination of the poxB gene were located upstream and downstream from the gene and obtained by PCR amplification using a primer set including poxB 5′ H3 (SEQ ID NO: 14) and DpoxB_up 3′ (SEQ ID NO: 15) and a primer set including DpoxB_dn 5′ (SEQ ID NO: 16) and poxB 3′ E1 (SEQ ID NO: 17). A pK19_ApoxB vector was prepared by cloning the obtained amplification product to the HindIII and EcoRI restriction enzyme positions of pK19 mobsacB vector.

Two homologous regions for the elimination of the pta-ackA gene were located upstream and downstream from the gene and obtained by PCR amplification using a primer set including pta 5′ H3 (SEQ ID NO: 18) and Dpta_up_R1 3′ (SEQ ID NO: 19) and a primer set including DackA_dn_R1 5′ (SEQ ID NO: 20) and ackA 3′ Xb (SEQ ID NO: 21). A pK19_Δpta_ackA vector was prepared by cloning the obtained amplification product to the HindIII and XbaI restriction enzyme positions of pK19 mobsacB vector.

Two homologous regions for the elimination of the actA gene were located upstream and downstream from the gene and obtained by PCR amplification using a primer set including actA 5′ Xb (SEQ ID NO: 22) and DactA_up_R4 3′ (SEQ ID NO: 23) and a primer set including DactA_dn_R4 5′ (SEQ ID NO: 24) and actA 3′ H3 (SEQ ID NO: 25). A pK19_ΔactA vector was prepared by cloning the obtained amplification product to the XbaI and HindIII restriction enzyme positions of pK19 mobsacB vector.

(2) Preparation of CGL (Δldh, ΔpoxB, Δpta-ackA, ΔactA)

The replacement vectors were introduced together to C. glutamicum ATCC13032 by electroporation. The strain in which the replacement vectors were introduced was cultured at 30° C. by streaking the strain on a lactobacillus selection (LBHIS) agar plate including kanamycin 25 ug/ml (micrograms per milliliter). The LBHIS agar plate included Difco LB™ broth 25 g/L, brain-heart infusion broth 18.5 g/L, D-sorbitol 91 g/L and agar 15 g/L. Hereinafter, the composition of the LBHIS agar plate is the same. Colonies on the agar plate were cultured at 30° C. in a BHIS medium (pH 7.0) including brain heart infusion powder 37 g/L and D-sorbitol 91 g/L. The culture was streaked on an LB/Suc10 agar plate and cultured at 30° C., and then only the colonies in which double crossing-over occurred were selected. The LB/Suc10 agar plate included Difco LB™ broth 25 g/L, agar 15 g/L, and sucrose 100 g/L.

After separating genomic DNA from the selected colonies, deletion of the genes was verified. Deletion of the ldh gene was verified through PCR using a primer set including IdhA_{—}5′_HindIII and IdhA_{—}3′_EcoRI, and deletion of the poxB gene was verified through PCR using a primer set including poxB_up_for (SEQ ID NO: 26) and poxB_dn_rev (SEQ ID NO: 27). In addition, deletion of the pta-ackA gene was verified through PCR using a primer set including pta_up_for (SEQ ID NO: 28) and ackA_dn_rev (SEQ ID NO: 29), and deletion of the actA gene was verified through PCR using a primer set including actA_up_for (SEQ ID NO: 30) and actA_dn_rev (SEQ ID NO: 31).

The strain was cultured in a method described in Example 3. As a result, a concentration of 61 g/L of succinic acid was produced within 134 hours following the conversion of the culture condition to anaerobic conditions. Analysis of intracellular metabolites of the strain showed that the quantity of malate was about eight times the size of the quantity of fumarate. Therefore, increasing the efficiency of converting malate to fumarate was assumed as being an important strategy in increasing succinic acid production.

### EXAMPLE 2

### Preparation of a Corynebacterium Microorganism, Wherein fumB Gene of Escherichia coli is Introduced

Corynebacterium fumarase fumC (SEQ ID NO: 3) was substituted with fumB (SEQ IN NO.1), which is a fumarase having a higher substrate affinity to malate than to fumarate among fumarase isoenzymes of Escherichia coli. Table 1 below shows the Kₘ values of fumarase A, B, and C to malate and to fumarate.

TABLE 1

| | |
|---|---|
| Type of | |
| Escherichia coli | Kₘ (mM) |

| Fumarase | malate | fumarate |
|---|---|---|
| fumA | 2.94 | 0.39 |
| fumB | 0.63 | 1.7 |
| fumC | 0.7 | 0.6 |

To delete the Corynebacterium fumC gene (SEQ ID NO: 4) and introduce the Escherichia coli fumB gene (SEQ ID NO: 2) simultaneously, a recombinant vector pK19_ΔfumC_P29::Ec.fumB was prepared on the basis of pK19 mobsacB vector (ATCC 87098). The recombinant vector was introduced to the strain prepared in Example 1, and the strain was streaked on an LBHIS agar plate including kanamycin 50 ug/ml and cultured at 30° C. Colonies on the agar plate were cultured at 30° C. in a BHIS medium (pH 7.0) having the composition described in Example 1. The culture was streaked on an LB/Suc10 agar plate having the composition described in Example 1 and cultured at 30° C., and then only the colonies in which double crossing-over occurred were selected. The genomic DNA was separated from the selected colonies. Deletion of the fumC gene and introduction of the fumB gene were verified through PCR by using a primer set including fumC_C_F (SEQ ID NO: 32) and fumC_C_R (SEQ ID NO: 33).

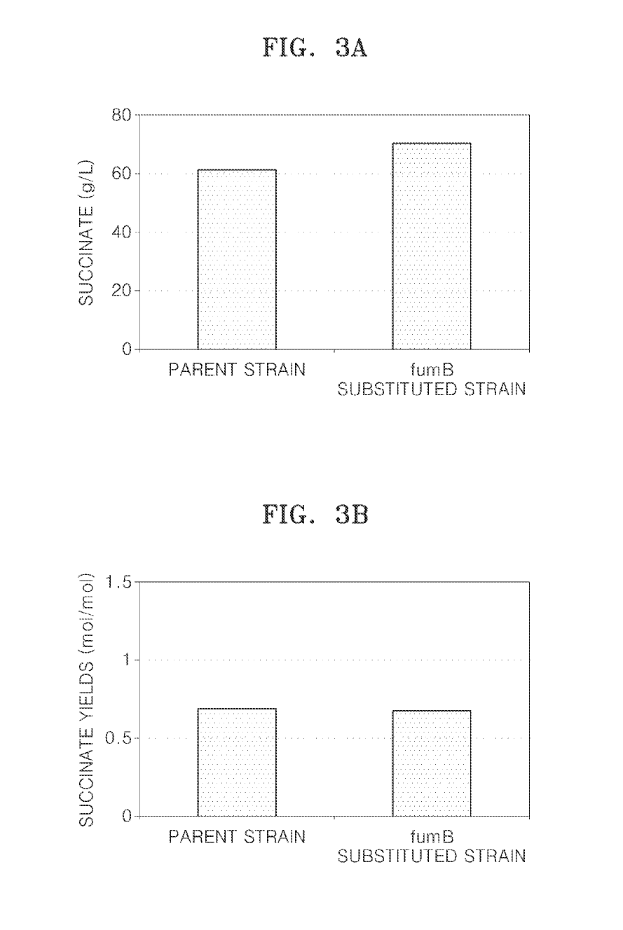

FIG. 2 illustrates a restriction map of pK19_ΔfumC_P29::Ec.fumB recombinant vector.

### EXAMPLE 3

### Comparison of Succinic Acid Productivity

Succinic acid productivity of the fumB-substituted strain obtained in Example 2 was compared with succinic acid productivity of the parent strain of Example 1 having the fumC gene.

For seed culture, each strain was streaked on an active plate including yeast extract 5 g/L, beef extract 10 g/L, polypeptone 10 g/L, NaCl 5 g/L, and agar 20 g/L, and then cultured at 30° C. for 48 hours. A single colony was seeded to a 5 ml S1 medium including 40 g/L glucose, 10 g/L polypeptone, 5 g/L yeast extract, 2 g/L (NH₄)₂SO₄, 4 g/L KH₂PO₄, 8 g/L K₂HPO₄, 0.5 g/L MgSO₄.H₂O, 1 mg/L thiamine-HCl, 0.1 mg/L D-biotin, 2 mg/L Ca-pantothenate, and 2 mg/L nicotineamide, and then cultured at 30° C. until the optical density value at 600 nanometers (OD₆₀₀) value became 5.0. The culture was transported to a 70 ml S1 medium and cultured at 30° C. for five hours.

The culture was started with a 700 ml culture in a 2.5 L fermenter. As a neutralizing agent, 5 mM NH₄OH was used. The seed culture was transported to an SF1 medium including 150 g/L glucose, 10 g/L corn-steep liquor, 2 g/L (NH₄)₂SO₄, 1 g/L KH₂PO₄, 0.5 g/L MgSO₄.H₂O, 10 mg/L FeSO₄.H₂O, 10 mg/L MnSO₄.H₂O, 0.1 mg/L ZnSO₄.H₂O, 0.1 mg/L CuSO₄.H₂O, 3 mg/L thiamine-HCl, 0.3 mg/L D-biotin, 1 mg/L Ca-pantothenate, and 5 mg/L nicotineamide. The culture solution was cultured at a stirring rate of 600 rpm and at a flow rate of 1.2 vvm until the OD₆₀₀ value became 120, and then cultured at a stirring rate of 200 rpm and at a flow rate of 0 vvm.

Samples were taken after culturing the culture solution for 134 hours under anaerobic conditions, and then the samples were centrifuged. The succinic acid and glucose concentrations of the supernatant were analyzed by HPLC.

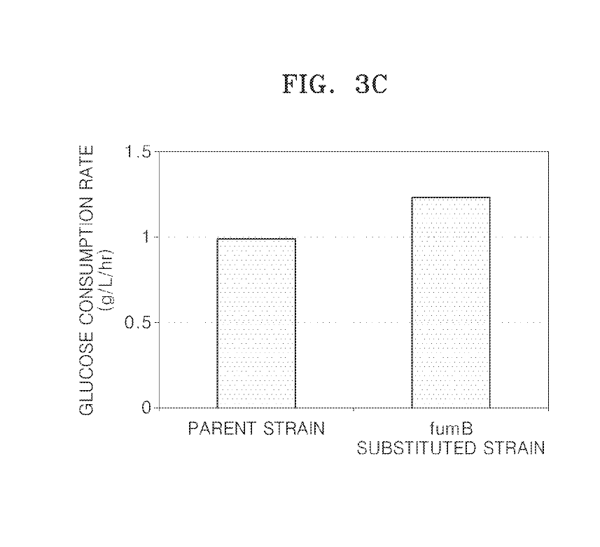

FIGS. 3A to 3C illustrate the culture results of a Corynebacterium microorganism wherein a fumC gene is substituted with a fumB gene. The fumB-substituted strain produced succinic acid of 70 g/L, which was 15% more than the succinic acid production of the parent strain. The fumB-substituted strain produced 0.68 mol succinic acid per 1 mol consumed glucose. In addition, the glucose consumption rate of the fumB-substituted strain was 25% higher than that of the parent strain.

As described above, the Corynebacterium genus microorganism according to one aspect of the present disclosure may be used for production of reductive metabolites.

C4 dicarboxylic acid may be efficiently produced by the method of producing C4 dicarboxylic acid according to another aspect of the present disclosure.

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A recombinant Corynebacterium genus microorganism comprising a gene encoding a fumarase polypeptide having a higher substrate affinity to malate than to fumarate.

2. The microorganism of claim 1, wherein the fumarase polypeptide is fumarase B.

3. The microorganism of claim 2, wherein the fumarase B comprises an amino acid sequence having a sequence identity of 95% or higher with SEQ ID NO:1.

4. The microorganism of claim 1, wherein the gene comprises a nucleotide sequence of SEQ ID NO: 2.

5. The microorganism of claim 1, wherein the recombinant microorganism does not have endogenous fumarase activity, or has decreased endogenous fumarase activity compared to a microorganism of the same type which is not genetically engineered.

6. The microorganism of claim 5, wherein any endogenous fumarase gene in the recombinant microorganism is inactivated or attenuated compared to a microorganism of the same type which is not genetically engineered.

7. The microorganism of claim 1, wherein activity of at least one protein selected from the group consisting of L-lactate dehydrogenase (LDH), pyruvate oxidase (PoxB), phosphotransacetylase (PTA), acetate kinase (AckA), and acetate coenzyme A transferase (ActA) is eliminated or decreased compared to a microorganism of the same type which is not genetically engineered.

8. The microorganism of claim 7, wherein at least one gene selected from the group consisting of a gene encoding LDH, a gene encoding PoxB, a gene encoding PTA, a gene encoding AckA, and a gene encoding ActA is inactivated or attenuated compared to a microorganism of the same type which is not genetically engineered.

9. The microorganism of claim 1, wherein the Corynebacterium genus microorganism is Corynebacterium glutamicum.

10. A method of producing a C4 dicarboxylic acid, comprising:
culturing the Corynebacterium genus microorganism of claim 1, whereby the microorganism produces a C4 dicarboxylic acid; and
recovering the C4 dicarboxylic acid from the culture.

11. The method of claim 10, wherein the microorganism is cultured under anaerobic conditions.

12. The method of claim 10, wherein the C4 dicarboxylic acid is succinic acid.

13. A method of producing the recombinant Corynebacterium genus microorganism of claim 1 comprising introducing into the Corynebacterium a gene encoding a fumarase polypeptide having a higher substrate affinity to malate than to fumarate.

14. The method of claim 13, wherein the introduction of the gene into the Corynebacterium is performed by homologous recombination.

15. The method of claim 13, wherein the method further comprises inactivating any endogenous fumarase gene in the recombinant microorganism.


SEQUENCE LISTING (33 sequences)

SEQ ID NO: 1 - protein, 548 aa, Escherichia coli
MSNKPFIYQAPFPMGKDNTEYYLLTSDYVSVADFDGETILKVEPEALTLLAQQAFHDASFMLRPAHQKQVAAILHDPEASENDKYVALQFLRNSEIAAKGVLPTCQDTGTAIIVGKKGQRVWTGGGDEETLSKGVYNTYIEDNLRYSQNAALDMYKEVNTGTNLPAQIDLYAVDGDEYKFLCVAKGGGSANKTYLYQETKALLTPGKLKNFLVEKMRTLGTAACPPYHIAFVIGGTSAETNLKTVKLASAHYYDELPTEGNEHGQAFRDVQLEQELLEEAQKLGLGAQFGGKYFAHDIRVIRLPRHGASCPVGMGVSCSADRNIKAKINREGIWIEKLEHNPGQYIPQELRQAGEGEAVKVDLNRPMKEILAQLSQYPVSTRLSLTGTIIVGRDIAHAKLKELIDAGKELPQYIKDHPIYYAGPAKTPAGYPSGSLGPTTAGRMDSYVDLLQSHGGSMIMLAKGNRSQQVTDACHKHGGFYLGSIGGPAAVLAQQSIKHLECVAYPELGMEAIWKIEVEDFPAFILVDDKGNDFFQQIVNKQCANCTK

SEQ ID NO: 2 - DNA, 1647 bp, Escherichia coli
atgtcaaacaaaccctttatctaccaggcacctttcccgatggggaaagacaataccgaatactatctactcacttccgattacgttagcgttgccgacttcgacggcgaaaccatcctgaaagtggaaccagaagccctgaccctgctggcgcagcaagcctttcacgacgcttcttttatgctccgcccggcacaccagaaacaggttgcggctattcttcacgatccagaagccagcgaaaacgacaagtacgtggcgctgcaattcttaagaaactccgaaatcgccgccaaaggcgtgctgccgacctgccaggataccggcaccgcgatcatcgtcggtaaaaaaggccagcgcgtgtggaccggcggcggtgatgaagaaacgctgtcgaaaggcgtctataacacctatatcgaagataacctgcgctattcacagaatgcggcgctggacatgtacaaagaggtcaacaccggcactaacctgcctgcgcaaatcgacctgtacgcggtagatggcgatgagtacaaattcctttgcgttgcgaaaggcggcggctctgccaacaaaacgtatctctaccaggaaaccaaagccctgctgactcccggcaaactgaaaaacttcctcgtcgagaaaatgcgtaccctcggtactgcagcctgcccgccgtaccatatcgcgtttgtgattggcggtacgtctgcggaaaccaacctgaaaaccgtcaagttagcaagcgctcactattacgatgaactgccgacggaagggaacgaacatggtcaggcgttccgcgatgtccagctggaacaggaactgctggaagaggcccagaaactcggtcttggcgcgcagtttggcggtaaatacttcgcgcacgacattcgcgttatccgtctgccacgtcacggcgcatcctgcccggtcggcatgggcgtctcctgctccgctgaccgtaacattaaagcgaaaatcaaccgcgaaggtatctggatcgaaaaactggaacacaacccaggccagtacattccacaagaactgcgccaggccggtgaaggcgaagcggtgaaagttgaccttaaccgcccgatgaaagagatcctcgcccagctttcgcaatacccggtatccactcgtttgtcgctcaccggcaccattatcgtgggccgagatattgcacacgccaagctgaaagagctgattgacgccggtaaagaacttccgcagtacatcaaagatcacccgatctactacgcgggtccggcgaaaacccctgccggttatccatcaggttcacttggcccaaccaccgcaggccgtatggactcctacgtggatctgctgcaatcccacggcggcagcatgatcatgctggcgaaaggtaaccgcagtcagcaggttaccgacgcgtgtcataaacacggcggcttctacctcggtagcatcggcggtccggcggcggtactggcgcagcagagcatcaagcatctggagtgcgtcgcttatccggagctgggtatggaagctatctggaaaatcgaagtagaagatttcccggcgtttatcctggtcgatgacaaaggtaacgacttcttccagcaaatcgtcaacaaacagtgcgcgaactgcactaagtaa

SEQ ID NO: 3 - protein, 469 aa, Corynebacterium glutamicum
MTEQEFRIEHDTMGEVKVPAKALWQAQTQRAVENFPISGRGLESAQIRAMGLLKAACAQVNKDSGALDAEKADAIIAAGKEIASGKHDAEFPIDVFQTGSGTSSNMNTNEVIASIAKANGVEVHPNDHVNMGQSSNDTFPTATHVAATEAAVNDLIPGLKVLHESLAKKANEWSEVVKSGRTHLMDAVPVTLGQEFGGYARQIQLGIERVEATLPRLGELAIGGTAAGTGINTSADFGGKVVAELINLTDVKELKEAENHFEAQAARDALVEFSGAMRVIAVSLYKIANDIRLMGSGPLTGLGEIRLPDLQPGSSIMPGKVNPVLCETATQVSAQVIGNDAAVAFSGTQGQFELNVFIPVMARNVLESARLLANTSRVFATRLVDGIEPNEAHMKELAESSPSIVTPLNSAIGYEAAAKVAKTALAEGKTIRQTVIDLGLVDGEKLTEEELDKRLDVLAMAHTERENKF

SEQ ID NO: 4 - DNA, 1410 bp, Corynebacterium glutamicum
atgaccgagcaggaattccgtattgagcacgacaccatgggtgaagtgaaggttccagcaaaggctctgtggcaggcacagacccagcgcgctgttgagaacttccctatctctggtcgtggtctggaatccgcacagatccgcgcaatgggtctgctgaaggcagcttgtgcgcaggtaaacaaggactccggtgcgctggatgcagagaaggcagatgccatcattgcagctggtaaggagatcgcgtccggtaagcatgacgctgagttcccaattgatgtgttccagactggttccggtacttcctccaacatgaacaccaatgaggttatcgcttccatcgcgaaggctaacggcgttgaggttcacccaaatgaccacgtcaacatgggtcagtcctccaatgacaccttccctactgcaactcacgttgctgcaaccgaagctgctgtcaatgacctcatcccaggcctgaaggttctgcacgagtctttggcgaagaaggctaacgagtggtctgaggttgttaagtccggccgcacccacctgatggacgctgttccagtaaccctgggccaggagttcggtggctacgctcgccagatccagctcggcatcgagcgcgttgaggctactcttcctcgccttggtgagctggctattggtggcaccgctgctggtaccggtatcaacacctccgctgatttcggcggcaaggttgttgctgaactgatcaacttgaccgacgtcaaggagctcaaggaagctgagaaccacttcgaggctcaggctgcacgcgacgctcttgttgagttctccggcgcaatgcgcgttatcgctgtctccttgtacaagatcgctaacgatatccgcctcatgggctccggcccactgaccggtcttggcgagatccgtctcccagacctgcagccaggttcctccatcatgccaggcaaggtcaacccagttctctgtgagaccgctacccaggtttccgctcaggttatcggcaatgacgcagctgttgcgttctccggcacccagggccagttcgagctcaacgtgttcatcccagtgatggctcgcaacgtgcttgagtccgctcgcctgctggctaacacttcccgcgtgttcgcaacccgtctcgttgatggcattgagccaaacgaggcacacatgaaggagctcgctgagtcttcaccttccatcgttaccccactgaactctgcaatcggctacgaagctgctgcaaaggtggctaagactgctttggctgagggcaagaccatccgccagactgtcatcgatttgggcttggttgatggcgagaagctcaccgaggaagagctggacaagcgcctcgacgttcttgctatggctcacaccgagcgcgagaacaagttctaa

SEQ ID NO: 5 - protein, 314 aa, Corynebacterium glutamicum
MKETVGNKIVLIGAGDVGVAYAYALINQGMADHLAIIDIDEKKLEGNVMDLNHGVVWADSRTRVTKGTYADCEDAAMVVICAGAAQKPGETRLQLVDKNVKIMKSIVGDVMDSGFDGIFLVASNPVDILTYAVWKFSGLEWNRVIGSGTVLDSARFRYMLGELYEVAPSSVHAYIIGEHGDTELPVLSSATIAGVSLSRMLDKDPELEGRLEKIFEDTRDAAYHIIDAKGSTSYGIGMGLARITRAILQNQDVAVPVSALLHGEYGEEDIYIGTPAVVNRRGIRRVVELEITDHEMERFKHSANTLREIQKQFF

SEQ ID NO: 6 - protein, 579 aa, Corynebacterium glutamicum
MAHSYAEQLIDTLEAQGVKRIYGLVGDSLNPIVDAVRQSDIEWVHVRNEEAAAFAAGAESLITGELAVCAASCGPGNTHLIQGLYDSHRNGAKVLAIASHIPSAQIGSTFFQETHPEILFKECSGYCEMVNGGEQGERILHHAIQSTMAGKGVSVVVIPGDIAKEDAGDGTYSNSTISSGTPVVFPDPTEAAALVEAINNAKSVTLFCGAGVKNARAQVLELAEKIKSPIGHALGGKQYIQHENPFEVGMSGLLGYGACVDASNEADLLILLGTDFPYSDFLPKDNVAQVDINGAHIGRRTTVKYPVTGDVAATIENILPHVKEKTDRSFLDRMLKAHERKLSSVVETYTHNVEKHVPIHPEYVASILNELADKDAVFTVDTGMCNVWHARYIENPEGTRDFVGSFRHGTMANALPHAIGAQSVDRNRQVIAMCGDGGLGMLLGELLTVKLHQLPLKAVVFNNSSLGMVKLEMLVEGQPEFGTDHEEVNFAEIAAAAGIKSVRITDPKKVREQLAEALAYPGPVLIDIVTDPNALSIPPTITWEQVMGFSKAATRTVFGGGVGAMIDLARSNIRNIPTP

SEQ ID NO: 7 - protein, 461 aa, Corynebacterium glutamicum
MSDTPTSALITTVNRSFDGFDLEEVAADLGVRLTYLPDEELEVSKVLAADLLAEGPALIIGVGNTFFDAQVAAALGVPVLLLVDKQGKHVALARTQVNNAGAVVAAAFTAEQEPMPDKLRKAVRNHSNLEPVMSAELFENWLLKRARAEHSHIVLPEGDDDRILMAAHQLLDQDICDITILGDPVKIKERATELGLHLNTAYLVNPLTDPRLEEFAEQFAELRKSKSVTIDEAREIMKDISYFGTMMVHNGDADGMVSGAANTTAHTIKPSFQIIKTVPEASVVSSIFLMVLRGRLWAFGDCAVNPNPTAEQLGEIAVVSAKTAAQFGIDPRVAILSYSTGNSGGGSDVDRAIDALAEARRLNPELCVDGPLQFDAAVDPGVARKKMPDSDVAGQANVFIFPDLEAGNIGYKTAQRTGHALAVGPILQGLNKPVNDLSRGATVPDIVNTVAITAIQAGGRS

SEQ ID NO: 8 - protein, 397 aa, Corynebacterium glutamicum
MALALVLNSGSSSIKFQLVNPENSAIDEPYVSGLVEQIGEPNGRIVLKIEGEKYTLETPIADHSEGLNLAFDLMDQHNCGPSQLEITAVGHRVVHGGILFSAPELITDEIVEMIRDLIPLAPLHNPANVDGIDVARKILPDVPHVAVFDTGFFHSLPPAAALYAINKDVAAEHGIRRYGFHGTSHEFVSKRVVEILEKPTEDINTITFHLGNGASMAAVQGGRAVDTSMGMTPLAGLVMGTRSGDIDPGIVFHLSRTAGMSIDEIDNLLNKKSGVKGLSGVNDFRELREMIDNNDQDAWSAYNIYIHQLRRYLGSYMVALGRVDTIVFTAGVGENAQFVREDALAGLEMYGIEIDPERNALPNDGPRLISTDASKVKVFVIPTNEELAIARYAVKFA

SEQ ID NO: 9 - protein, 250 aa, Corynebacterium glutamicum
MSHMINKSISSTAEAVADIPDGASIAVGGFGLVGIPTALILALREQGAGDLTIISNNLGTDGFGLGLLLLDKKISKSIGSYLGSNKEYARQYLEGELTVEFTPQGTLAERLRAGGAGIPAFYTTAGVGTQVAEGGLPQRYNTDGTVAVVSQPKETREFNGQLYVMEEGIRADYALVHAHKADRFGNLVFRKTAQNFNPDAAMSGKITIAQVEHFVDELHPDEIDLPGIYVNRVVHVGPQETGIENRTVSN

SEQ ID NO: 10 - DNA, 43 bp, Artificial Sequence
  Synthetic (ldhA_5′_HindIII)
catgattacgccaagcttgagagcccaccacattgcgatttcc

SEQ ID NO: 11 - DNA, 42 bp, Artificial Sequence
  Synthetic (ldhA_up_3′_XhoI)
tcgaaactcgagtttcgatcccacttcctgatttccctaacc

SEQ ID NO: 12 - DNA, 39 bp, Artificial Sequence
  Synthetic (ldhA_dn_5′_XhoI)
tcgaaactcgagtaaatctttggcgcctagttggcgacg

SEQ ID NO: 13 - DNA, 46 bp, Artificial Sequence
  Synthetic (ldhA_3′_EcoRI)
acgacggccagtgaattcgacgacatctgagggtggataaagtggg

SEQ ID NO: 14 - DNA, 42 bp, Artificial Sequence
  Synthetic (poxB 5′ H3)
catgattacgccaagctttcagcgtgggtcgggttctttgag

SEQ ID NO: 15 - DNA, 32 bp, Artificial Sequence
  Synthetic (DpoxB_up 3′)
aatcatcatctgaactcctcaacgttatggct

SEQ ID NO: 16 - DNA, 37 bp, Artificial Sequence
  Synthetic (DpoxB_dn 5′)
ggagttcagatgatgattgatacacctgctgttctca

SEQ ID NO: 17 - DNA, 44 bp, Artificial Sequence
  Synthetic (poxB 3′ E1)
acgacggccagtgaattcatgtcccgaatccacttcaatcagag

SEQ ID NO: 18 - DNA, 44 bp, Artificial Sequence
  Synthetic (pta 5′ H3)
catgattacgccaagcttccctccatgatacgtggtaagtgcag

SEQ ID NO: 19 - DNA, 43 bp, Artificial Sequence
  Synthetic (Dpta_up_R1)
gttccctgttaatgtaaccagctgaggtcggtgtgtcagacat

SEQ ID NO: 20 - DNA, 48 bp, Artificial Sequence
  Synthetic (DackA_dn_R1)
ttacattaacagggaaccggaagagttagctatcgctaggtacgcggt

SEQ ID NO: 21 - DNA, 40 bp, Artificial Sequence
  Synthetic (ackA 3′ Xb)
acccggggatcctctagagggctgatgtgatttctgcggg

SEQ ID NO: 22 - DNA, 41 bp, Artificial Sequence
  Synthetic (actA 5′ Xb)
ggtggcggccgctctagaggtctgagctttattcctgggct

SEQ ID NO: 23 - DNA, 36 bp, Artificial Sequence
  Synthetic (DactA_up_R4)
tctggatagaagcatctaagccagcgccggtgaagc

SEQ ID NO: 24 - DNA, 46 bp, Artificial Sequence
  Synthetic (DactA_dn_R4)
agatgcttctatccagagctccggtgacaacaagtacatgcagacc

SEQ ID NO: 25 - DNA, 39 bp, Artificial Sequence
  Synthetic (actA 3′ H3)
gacggtatcgataagcttcgtacgatgcttgagcggtat

SEQ ID NO: 26 - DNA, 19 bp, Artificial Sequence
  Synthetic (poxB_up_for)
ggctgaaaccaaaccagac

SEQ ID NO: 27 - DNA, 22 bp, Artificial Sequence
  Synthetic (poxB_dn_rev)
ctgcatgatcggttagatacag

SEQ ID NO: 28 - DNA, 18 bp, Artificial Sequence
  Synthetic (pta_up_for)
gcgtggaattgagatcgg

SEQ ID NO: 29 - DNA, 18 bp, Artificial Sequence
  Synthetic (ackA_dn_rev)
cagagcgatttgtggtgg

SEQ ID NO: 30 - DNA, 20 bp, Artificial Sequence
  Synthetic (actA_up_for)
tgaagcaatggtgtgaactg

SEQ ID NO: 31 - DNA, 19 bp, Artificial Sequence
  Synthetic (actA_dn_rev)
gctaccaaacactagcctg

SEQ ID NO: 32 - DNA, 21 bp, Artificial Sequence
  Synthetic (fumC_C_F)
ggagatcatgaagtttcgtgg

SEQ ID NO: 33 - DNA, 20 bp, Artificial Sequence
  Synthetic (fumC_C_R)
cttcgtcgacgctgaatcag